(19) United States
(12) Patent Application Publication
(10) Pub. No.: US 2017/0214269 A1
(43) Pub. Date: Jul. 27, 2017

(54) Physically-Configurable External Charger for an Implantable Medical Device with Receptacle in Coil Housing for Electronics Module

(71) Applicant: Boston Scientific Neuromodulation Corporation, Valencia, CA (US)

(72) Inventor: Joshua D. Howard, Winnetka, CA (US)

(21) Appl. No.: 15/354,444

(22) Filed: Nov. 17, 2016

Related U.S. Application Data
(60) Provisional application No. 62/286,257, filed on Jan. 22, 2016.

(51) Int. Cl. H02J 7/02; A61N 1/372

(52) U.S. Cl. CPC H02J 7/025; H02J 7/027; A61N 1/372

(57) ABSTRACT

A physically-configurable external charger device for an implantable medical device is disclosed, which facilitates the generation of different powers of a magnetic field with reduced heating concerns. A housing which includes an internal charging coil includes a receptacle for holding an electronics module for energizing the charging coil. A cable coupled to the charging coil spans around the edges of the housing and connects to the electronics module when it is retained by the receptacle. In this first configuration, a low-power magnetic field can be produced, as the electronics module is still relatively near the charging coil, and thus may heat to some degree. In a second configuration, the electronics module is removed from the receptacle and extendable from the housing by the length of the cable, and thus a higher-power magnetic field can be produced with reduced heating concerns.

### CROSS REFERENCE TO RELATED APPLICATIONS

This is a non-provisional of U.S. Provisional Patent Application Ser. No. 62/286,257, filed Jan. 22, 2016, to which priority is claimed, and which is incorporated herein by reference in its entirety.

This application is also related to U.S. Provisional Patent Application Ser. No. 62/286,253, filed Jan. 22, 2016.

### FIELD OF THE INVENTION

The present invention relates to a wireless charger for an implantable medical device such as an implantable pulse generator.

### BACKGROUND

Implantable stimulation devices are devices that generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system.

As shown in FIGS. 1A and 1B, a SCS system typically includes an Implantable Pulse Generator (IPG) 10, referred to more generically as an Implantable Medical Device (IMD) 10. IMD 10 includes a biocompatible device case 12 formed of a metallic material such as titanium for example. The case 12 typically holds the circuitry and battery 14 necessary for the IMD 10 to function, although IMDs can also be powered via external RF energy and without a battery, as described further below. The IMD 10 is coupled to electrodes 16 via one or more electrode leads (two such leads 18 are shown), such that the electrodes 16 form an electrode array 20. The electrodes 16 are carried on a flexible body 22, which also houses the individual signal wires 24 coupled to each electrode. In the illustrated embodiment, there are eight electrodes on each lead, although the number of leads and electrodes is application specific and therefore can vary. The leads 18 couple to the IMD 10 using lead connectors 26, which are fixed in a header 28 comprising epoxy for example, which header is affixed to the case 12. In a SCS application, distal ends of electrode leads 18 with the electrodes 16 are typically implanted on the right and left side of the dura within the patient's spinal cord. The proximal ends of leads 18 are then tunneled through the patient's tissue to a distant location such as the buttocks where the IMD 10 is implanted, where the proximal leads ends are then connected to the lead connectors 26.

As shown in cross section in FIG. 2B, the IMD 10 typically includes a printed circuit board (PCB) 30 containing various electronic components 32 necessary for operation of the IMD 10. Two coils are present in the IMD 10 as illustrated: a telemetry coil 34 used to transmit/receive data to/from an external controller (not shown); and a charging coil 36 for receiving power from an external charger 40 (FIG. 2A). These coils 34 and 36 are also shown in the perspective view of the IMD 10 in FIG. 1B, which omits the case 12 for easier viewing. Although shown as inside in the case 12 in the Figures, the telemetry coil 34 can alternatively be fixed in header 28. Coils 34 and 36 may alternative be combined into a single telemetry/charging coil.

FIG. 2A shows a plan view of the external charger 40, and FIG. 2B shows it in cross section and in relation to the IMD 10 as it provides power—either continuously if the IMD 10 lacks a battery 14, or intermittently if the charger is used during particular charging sessions to recharge the battery. In the depicted example, external charger 40 includes two PCBs 42a and 42b; various electronic components 44 for implementing charging functionality; a charging coil 46; and a battery 48 for providing operational power for the external charger 40 and for the production of a magnetic field 60 from the charging coil 46. These components are typically housed within a housing 50, which may be made of hard plastic such as polycarbonate for example.

The external charger 40 has a user interface 54, which typically comprises an on/off switch 56 to activate the production of the magnetic field 60; an LED 58 to indicate the status of the on/off switch 56 and possibly also the status of the battery 48; and a speaker (not shown). The speaker emits a “beep” for example if the external charger 40 detects that its charging coil 46 is not in good alignment with the charging coil 36 in the IMD 10. More complicated user interfaces 54 can be used as well, such as those involving displays or touch screens, or involving realistic audio output (e.g., speech or music) beyond a mere beep, etc.

The external charger's housing 50 is sized such that the external charger 40 is hand-holdable and portable. In an SCS application in which the IMD 10 is implanted behind the patient, the external charger 40 may be placed in a pouch (not shown) around a patient's waist to position the external charger in alignment with the IMD 10. Typically, the external charger 40 is touching the patient's tissue 70 as shown (FIG. 2B), although the patient's clothing or the material of the pouch may intervene.

Wireless power transfer from the external charger 40 to the IMD 10 occurs by near-field magnetic inductive coupling between coils 46 and 36. When the external charger 40 is activated (e.g., on/off switch 56 is pressed), charging coil 46 is driven with an AC current to create the magnetic field 60. The frequency of the magnetic field 60 may be on the order of 80 kHz for example, and may generally be set by the inductance of the coil 46 and the capacitance of a tuning capacitor (not shown) in the external charger 40. The magnetic field 60 transcutaneously induces an alternating current in the IMD 10's charging coil 36, which current is rectified to DC levels and used to power circuitry in the IMD 10 directly and/or to recharge the battery 14 if present.

The IMD 10 can communicate relevant data back to the external charger 40, such as the capacity of the battery using Load Shift Keying, as explained for example in U.S. Pat. Application Publication 2015/0077050, or by any other means. For example, either or both of the charging coil 36 or the telemetry coil 34 can be used to transmit data, or other separate data antennas (e.g., short-range far-field RF antennas, communicating by Bluetooth, WiFi, Zigbee, MICS, or other protocols) can be used in either or both of the IMD 10 and the external charger 40.

Referring again to FIG. 2B, the depicted example of the external charger 40 includes two PCBs 42a and 42b, which are generally orthogonal. The bulk of the electronic components 44 are carried on the vertical PCB 42b. Horizontal PCB 42a by contrast is generally free of components, and carries only the charging coil 46. Further, the battery 48 is placed outside of the area extent of the charging coil 46. As explained in U.S. Pat. No. 9,002,445, such design of the external charger 40 is useful to reduce heating, in particular heating of conductive components resulting from Eddy currents caused by the alternating magnetic field 60. The design moves conductive materials (the PCB 42b with its electronic components 44; the battery 48 with its conductive housing) away from where the magnetic field 60 is most intense in the center of the charging coil 46, as illustrated by the concentration of magnetic field flux lines, shown in dotted lines in FIG. 2C. Further, placing the electronic components 44 on a vertical PCB 42b tends to orient the major planes of the PCB 42b and components 44 parallel to the highest-intensity portions of the magnetic field 60 in the center of the coil 46, rendering such components that much less susceptible to Eddy current heating. The design of the external charger 40 is thus able to remain compact within its hand-holdable housing 50 without significant heating concerns.

Even if heating of the external charger 40 is mitigated by these design choices, it is still prudent to monitor temperature to ensure that a patient will not be injured while charging his IMD 10. In this regard, external charger 40 preferably includes at least one temperature sensor, such as a thermistor 52 (FIG. 2B), to monitor the external charger 40's temperature while charging. Thermistor 52 is preferably placed on the inside surface of the housing 50 that faces (and potentially touches) the patient when the external charger 40 is producing the magnetic field 60.

The thermistor 52 can communicate temperature to control circuitry (part of electronic components 44) within the external charger 70, to ensure that a maximum safe temperature for the patient, Tmax (e.g., 41° C.), is not exceeded. If the thermistor 52 reports this maximum temperature, and particularly in the circumstance where the external charger 40 is used to recharge an IMD 10's battery 14, charging may be suspended by ceasing current through the charging coil 46 to allow the external charger 40 to cool. Once cool enough, for example once the temperature drops to a lower minimum temperature, Tmin (e.g., 39° C.), charging may again be enabled by reinitiating the current through the charging coil 46, until Tmax is again reached and charging suspended, etc. This is illustrated in FIG. 3, and borrowed from U.S. Pat. No. 8,321,029. The patient may not be aware that the external charger 40 is actually duty cycling between enabled and suspended states to maintain a safe temperature during a battery charging session. Other means of temperature control beyond duty cycling exist, such as adjusting the magnitude of the current through the charging coil 46, detuning the frequency of the magnetic field 60, etc.

While external charger 40 works fine to provide power to a patient's IMD 10, the inventor sees room for improvement in external charger design. For example, the inventor notes that while the design of external charger 40 reduces Eddy-current-related heating by moving and orienting components as described above, Eddy current heating will still exist to some degree. As FIG. 2C shows, while the amount of magnetic flux impinging upon the vertically-oriented electronic components 44 and the battery 48 may be lessened, such components are still relatively close to the charging coil 46, and hence still receive magnetic field 60 and will heat to some degree.

The propensity of external charger 40 to heat ultimately impedes its ability to provide significant power to the IMD 10, or to quickly charge the IMD 10's battery 14. This is because Tmax effectively limits the strength of the magnetic field 60 that can be produced, and hence limits the rate at which the battery 14 can be charged.

Further, the inventor considers it unfortunate that the external charger 40 is formed as a single integrated unit. If just one portion of the external charger is malfunctioning (e.g., the charging coil 46, some electronic components 44, the battery 48, etc.), the entire external charger 40 will likely need to be replaced even though other portions may be working suitably. Likewise, the integrated design of the external charger 40 impedes the ability to upgrade its various portions with improved technology, even if such portions are otherwise working normally.

In recognition of these concerns, the inventor proposes a new external charger design that includes separable portions and is also physically configurable. A first physical configuration allows for low-power charging as described to this point, while a second physical configuration allows for high-powered charging, and hence faster IMD battery charging.

### BRIEF DESCRIPTION OF THE DRAWINGS

FIGS. 1A and 1B show an Implantable Medical Device (IMD), in accordance with the prior art.

FIGS. 2A-2C show an external charger for an IMD, in accordance with the prior art.

FIG. 3 shows means for controlling the temperature of the external charger during an IMD battery charging session, in accordance with the prior art.

FIGS. 4A and 4B show an improved external charger in top and side views respectively, and in a first physical configuration in which an electronics module is retained in a housing including the charging coil, in accordance with an example of the invention.

FIG. 5 shows the external charger in a second physical configuration in which the electronics module is extended from the housing by a cable, in accordance with an example of the invention.

FIG. 6A shows further details of the external charger, while FIGS. 6B-6D show various cross sections of the external charger, in accordance with an example of the invention.

FIG. 7 shows the electronics module, and specifically its circuitry module and its battery module, in accordance with an example of the invention.

FIGS. 8A and 8B show respective top and cross-sectional views of another example of the external charger having a means for holding the cable when the electronics module is retained within the housing, in accordance with an example of the invention.

FIGS. 9A and 9B show different orientations for the external charger's circuitry and its cable connector, in accordance with examples of the invention.

FIG. 10 shows alternative manners of positioning user interface elements on the electronics module of the external charger, in accordance with an example of the invention.

FIGS. 11A and 11B show use of the external charger to produce low- and high-power magnetic fields for an IMD in conjunction with a charging belt, in accordance with examples of the invention.

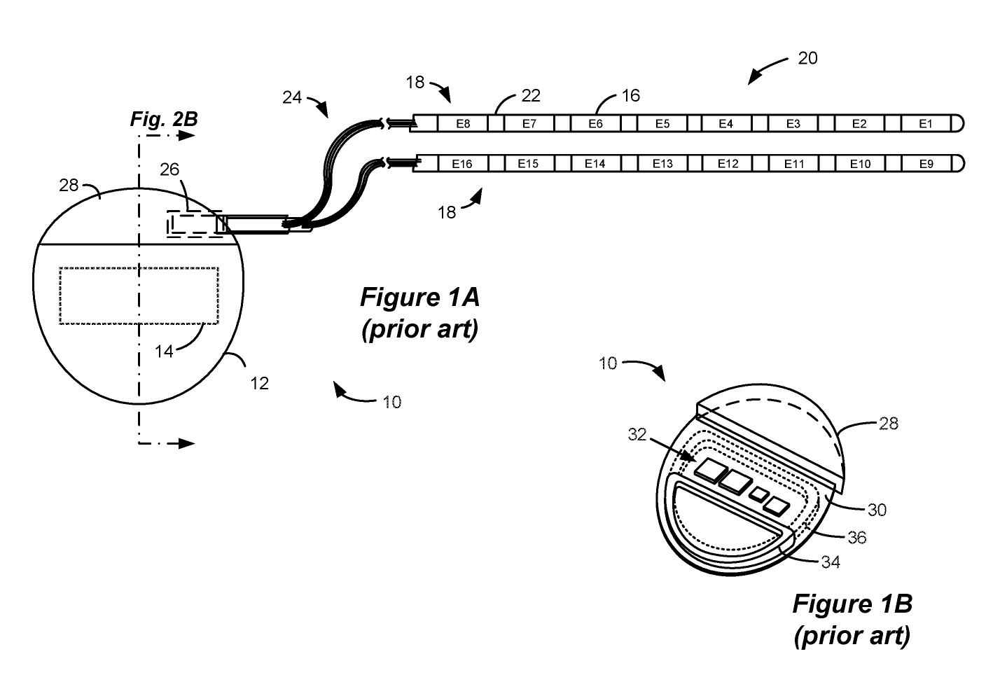

### DETAILED DESCRIPTION

A physically-configurable external charger device for an Implantable Medical Device (IMD) is disclosed, which facilitates the generation of different powers of a magnetic field but with reduced heating concerns at higher powers. A housing which includes an internal charging coil includes a receptacle for holding an electronics module for energizing the charging coil. A cable coupled to the charging coil spans around the edges of the housing and connects to the electronics module when it is retained by the receptacle, preferably by a connector/port arrangement. In this first physical configuration, a relatively low-power magnetic field can be produced, as the electronics module is still relatively near the charging coil (although outside of its area), and thus may heat to some degree. In a second physical configuration, the electronics module is removed from the receptacle and extended from the housing preferably by the length of the cable, and thus a higher-power magnetic field can be produced with reduced heating concerns. Thus, in this second configuration, the charging rate of the IMD can be increased. The design of the external charger is also modular, as the electronics module can be separable from the housing, and because circuitry and battery modules in the electronics module can be separable. This allows for easy replacement of portions of the external charger should one portion fail or need to be upgraded.

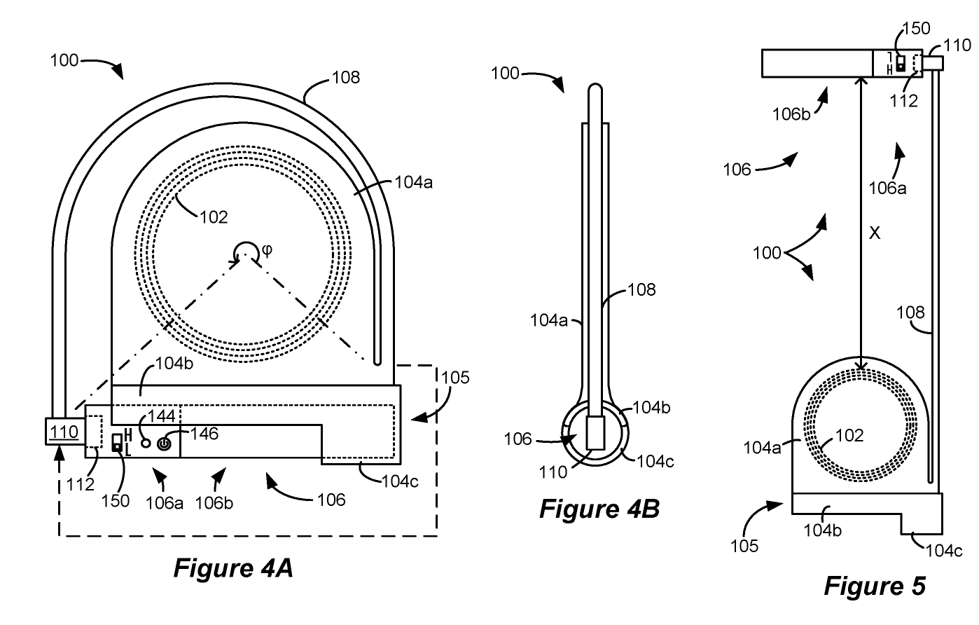

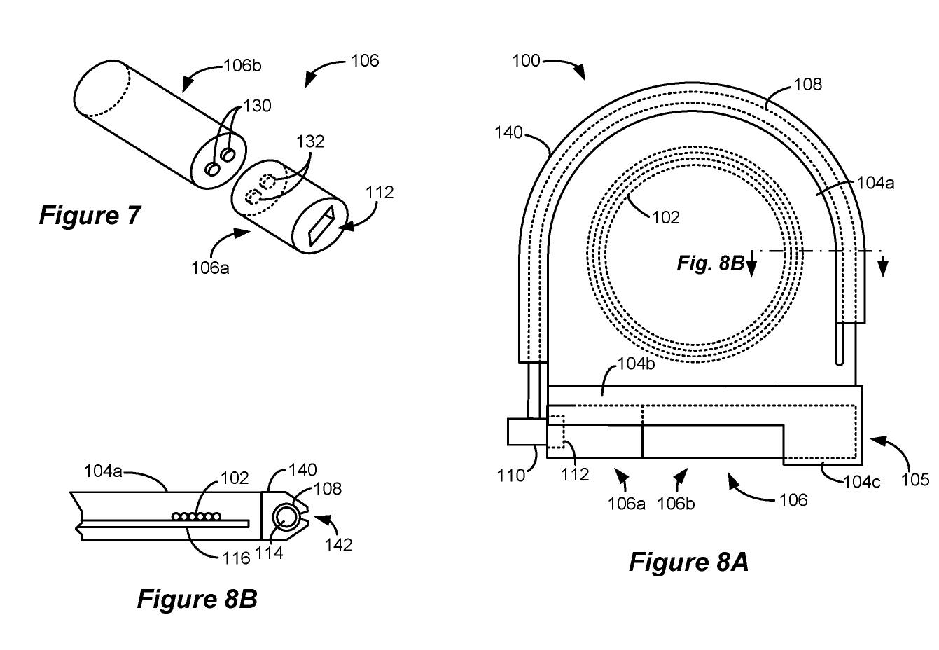

An example of an improved, physically-configurable external charger 100 is shown first in FIGS. 4A and 4B, which respectively show the charger from the top and side. The external charger 100 includes a housing 104 which as shown comprises three portions. A flat charging coil housing 104a includes a charging coil 102, which like the prior art charger is energized to produce a magnetic field 60 to power and/or charge the IMD 10. Further housing portions 104b and 104c are used to retain an electronics module 106, as explained below. The electronics module 106 is preferably split into two portions, namely a circuitry module 106a and a battery module 106b, as also explained below. In the depicted example, the electronics module 106 is cylindrical in shape (see FIG. 7), although this isn't necessary. For example, the electronics module 106 (i.e., either or both of 106a and 106b) could also be rectangular, triangular, elliptical, etc.

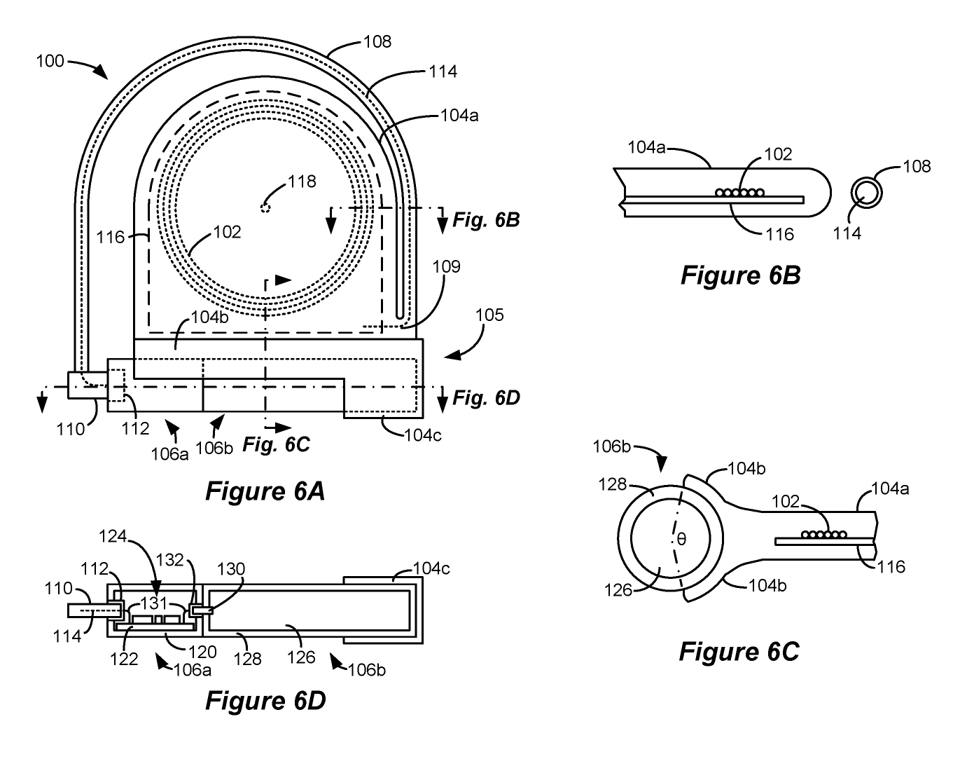

Housing portions 104b and 104c are configured to retain and release the electronics module 106 and are preferably formed to match the electronics module 106's shape. In particular, housing portion 104c comprises a cup into which an end of the electronics module 106 (e.g., battery module 106b) can be pressed (see FIG. 6D). Housing portion 104b is largely open to allow insertion and removal of the electronics module 106, and only partially surrounds the module. Housing portion 104b in the depicted example comprises a curved wall shaped to mate with part of the electronics module 106's curved outer surface. As shown in FIG. 6C, the curved wall 104b may span less than 180 degrees (θ) of the electronics module 106's curved surface. As such, curved wall 104b generally stabilizes the electronics module 106 when it is retained within the cup 104c, but still allows the electronics module 106 to be easily removed from the cup 104c. However, curved wall 104b may be greater than 180 degrees, allowing the electronics module 106 to be further secured, such as by snapping it into place within the curved wall 104b when the electronic module is retained, and snapping it back out when the electronics module 106 is removed. Together, either or both of housing portions 104b and 104c can be referred to generically as a “receptacle” 105, and in this regard receptacle 105 may include any means for releasably retaining the electronics module 106, and need not have cups or curved walls. For example, receptacle 105 could include clips, a groove, etc.

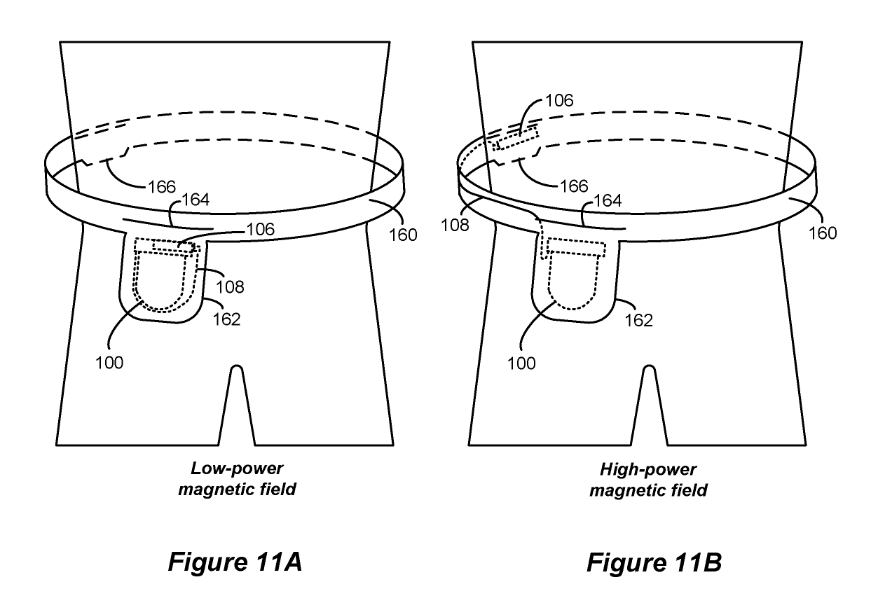

Housing portions 104a, 104b, and 104c may comprise a hard rubberized material or a polyurethane which are mold injected and hence formed as an integral piece. Note that because the coil housing 104a contains only minimal electronics, as described later, it can be made relatively thin compared to the thickness of the electronics module 106, as best shown in the side view of FIG. 4B. The thinness of the coil housing 104a is beneficial because its low profile is less conspicuous when used by a patient to charge his IMD 10, as explained further later with reference to FIGS. 11A and 11B. However, housings 104a, 104b, and 104c can be formed in other ways, such as of separate parts or of different materials.

As noted, electronics module 106 is preferably formed as two separate modules: a circuitry module 106a and a battery module 106b. Circuitry module 106a includes electronics components 124 (FIG. 6D) necessary for external charger operation, while battery module 106b includes a battery 126 (FIG. 6D) to power such electronics. Modules 106a and 106b are preferably attachable to and detachable from each other, and in this regard a connector/port arrangement may be used to secure them together. For example, and as shown in FIG. 7, battery connectors (terminals) 130 on the battery module 106b may be secured at ports 132 on the circuitry module 106a to allow the battery module 106b to provide power to the circuitry module 106a.

Battery 126 within the battery module 106b is depicted in the cross-section of FIG. 6D as having its own housing 128. Housing 128 may comprise the battery's prefabricated housing, which is typically conductive. Alternatively, housing 128 may comprise an additional housing into which an otherwise completed battery is placed, in which case housing 128 would preferably be insulating, similar to housing 120 of the circuitry module 106a. Battery 126 may be either non-rechargeable (primary) or rechargeable (e.g., a Li-ion polymer battery). If battery 126 is rechargeable, it may be recharged via port 112 of the circuitry module 106a, and in this regard electronic components 124 within the circuitry module 106a can include battery recharging circuitry, such as is disclosed in U.S. Patent Application Serial No. 2016/0126771.

Having separable circuitry 106a and battery 106b modules is preferable as it allows one or the other to be replaced. For example, battery module 106b can be replaced if battery 126 is either depleted (if non-rechargeable) or will no longer hold an adequate charge (if rechargeable). Likewise, circuitry module 106a can be replaced if it is malfunctioning. Replacements for either module 106a or 106b can include more advanced technology, for example, improved circuitry or a higher capacity battery 126. This being said, it is not required that circuitry and battery modules 106a and 106b be separable. Instead, they can be combined into a single electronics module 106 with a common housing 120, as shown in FIG. 10, which is explained later.

Referring again to FIG. 4A, a cable 108 connects electronics in the coil housing 104a such as the coil 102 to the circuitry module 106a (or electronics module 106 more generally). To assist in this connection, the end of cable 108 includes a connector 110 attachable to a port 112 (see also FIG. 7) on the flat face of the circuitry module 106a. Notice that the cable 108 spans around a portion of the edge of the coil housing 104a when the electronic module 106 is retained within the receptacle 105. The cable 108 preferably spans around an edge of the housing 104 (e.g., the coil housing 104a) which is different from the edge where the electronics module 106/receptacle 105 is located. In this regard, the cable 108 preferably spans approximately 270 degrees (φ) around the housing 104. Given possible differences in which external charger 100 can be fabricated, “approximately 270 degrees” should be understood as ranging from 180 degrees to 360 degrees. Further, an edge of the housing 104 need not be linear, but can comprise curved edges as well.

Spanning the cable 108 around the housing 104 is preferred both because it renders an organized and compact design when the electronics module 106 is retained in the receptacle 105, and because it yields a cable 108 of sufficient length X (FIG. 5) to position the electronics module 106 sufficiently far away from the charging coil 102 when it is removed from the receptacle 105, such as during high-power charging, and as illustrated in FIG. 5, explained further later. That being said, it is not strictly necessary that cable 108 proceed around the coil housing 104a or in a counter clockwise direction as shown. Instead, the cable 108 can alternatively proceed around the housing 104 in a clockwise direction, as shown by a dotted line in FIG. 4A.

Cable 108 includes inner wires 114 (FIGS. 6A & 6B) as necessary to connect electronics in the coil housing 104a to electronics in the circuitry module 106a. In this regard, coil housing 104a preferably includes a printed circuit board 116, as seen in FIGS. 6A-6C to support the charging coil 102 and any other electronics in the coil housing 104a. For example, the coil housing 104a may include at least one thermistor 118 (FIG. 6A) to report temperature to electronic components 124 in the circuitry module 106a. As shown, the thermistor 118 is preferably centered with respect to the charging coil 102. Printed circuit board 116 can be rigid (FR4), or of a flexible type such as Kapton™. Coil housing 104a may include other circuitry as well, such as driver circuitry for the charging coil 102, and thus while cable 108 may be coupled to the charging coil 102 via such other circuitry or connections, cable 108 is not necessarily connected directly to the charging coil 102.

The connector 110 type used with cable 108 should be chosen in light of how many wires 114 are required to adequately communicate between the various electronics in the coil housing 104a and the circuitry module 106a. In this regard, the connector 110/port 112 can comprise a mini HDMI port, a mini USB port, and the like, or may be customized.

Cable 108 and its connector 110 are attachable to and detachable from the electronics module 106, preferably the circuitry module 106a. This is preferred because (like the separability of circuitry module 106a and battery module 106b) it allows defective or out-of-date components in the external charger 100 to be replaced. For example, if the charging coil 102 in coil housing 104a continues to function appropriately, it may be retained while either or both of circuitry module 106a or battery module 106b are replaced. Similarly, either or both of circuitry module 106a or battery module 106b can be retained while coil housing 104a is replaced, which might occur either because coil 102 is defective (e.g., open circuited), or simply to provide a newer coil 102/housing 104a that might be of a different size and/or a more efficient design. This being said, connector 110 and port 112 in the electronics module 106 (circuitry module 106a) may alternatively be hardwired and not separable.

Cable 108 is preferably bendable to allow the electronics module 106 to be both retained within (FIG. 4A) and extended from (FIG. 5) the housing 104. In one example, the covering of the cable 108 may comprise a rubberized material, which along with its connector 110 can be mold injected along with one or more of the housing portions 104a-c. If housings 104a-c are made of harder materials, cable 108 may have a more softer covering similar to charging cables used with mobile devices generally. Although not shown, cable 108 can further include a stiffening member throughout its length, such as a bendable metal material that allows the cable to retain its shape when bent. This would allow the electronics module 106 when extended (FIG. 5) to independently retain its position relative to the housing 104. Although not shown, cable 108 may comprise at the opposite end from connector 110 a discrete attachment 109 (FIG. 6A) to the coil housing 104a. This attachment 109 may be hardwired, or may comprise a connector/port arrangement that allows cable 108 to be attached to and detached from the coil housing 104a.

If cable 108 is softer and “floppy,” it may be desirable to retain it against the edge of the coil housing 104a when the electronics module 106 is retained (FIG. 4A). In this regard, the edge of the coil housing 104a can include a cable-holding mechanism 140, as shown in FIGS. 8A and 8B. In this example, cable-holding mechanism 140 comprises a deformable rubberized material including a groove 142 into which the cable 108 can be press fit when the electronics module 106 is retained within receptacle 105 (FIG. 8A), and from which the cable 108 can be “peeled” when the electronics module 106 is removed from the receptacle 105 and extended from the housing 104 (FIG. 5). Although cable-holding mechanism 140 is shown in FIGS. 8A and 8B as comprising a material separate from the coil housing 104a, in other examples it could simply comprise the edge of the coil housing 104a as it is formed. Also, cable-holding mechanism 140 could comprise other well-known structures such as clips, clasps, Velcro™, etc. Further, cable-holding mechanism 140 can retain the cable 108 at discrete locations around the edge of the coil housing 104a, rather than retaining the cable along the continuum of the edge of the coil housing 104a as illustrated in FIG. 8A.

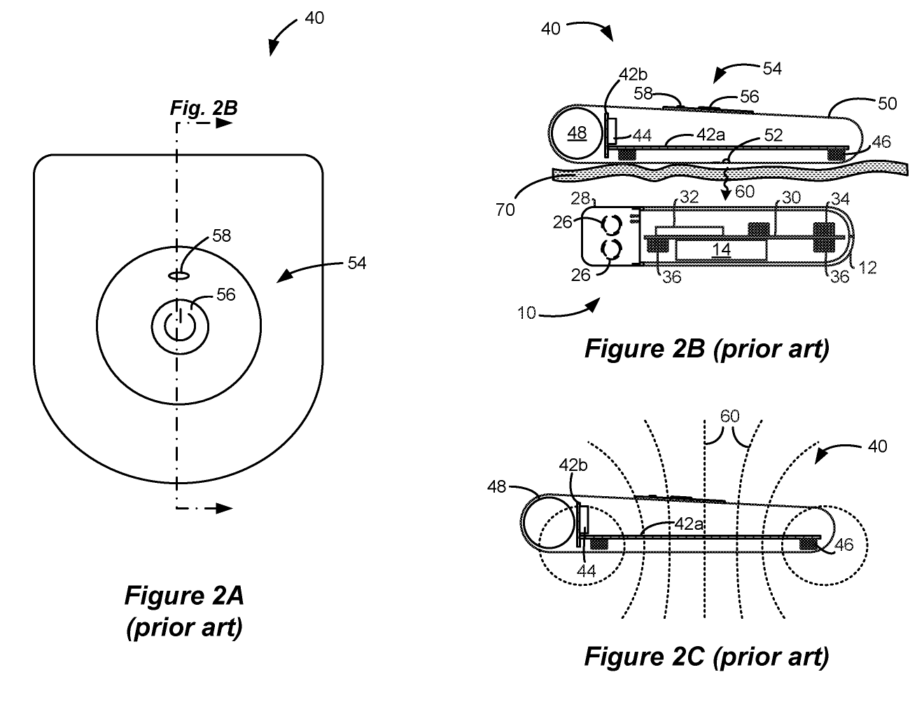

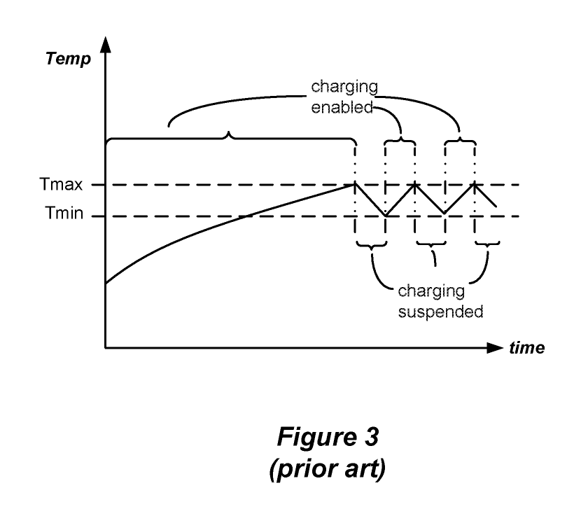

As best shown in the cross-section of FIG. 6D, circuitry module 106a preferably includes a printed circuit board 122 for integrating electronic components 124 to enable external charger 100 to operate to provide power/charging to an IMD 10. In this regard, electronic components 124 can be identical or similar to electronic components 44 otherwise generally included in traditional external chargers, such as external charger 40 of the prior art (FIGS. 2A-2C), and general functionality and control of external charger 100 can be the same, except as further described herein. As further shown in FIG. 6D, circuitry module 106a can receive power from the battery 126 (connectors 132/ports 130) and coil-housing related signals from the coil housing 104a (connector 110/port 112) via connections 131 that connect to the PCB 122.

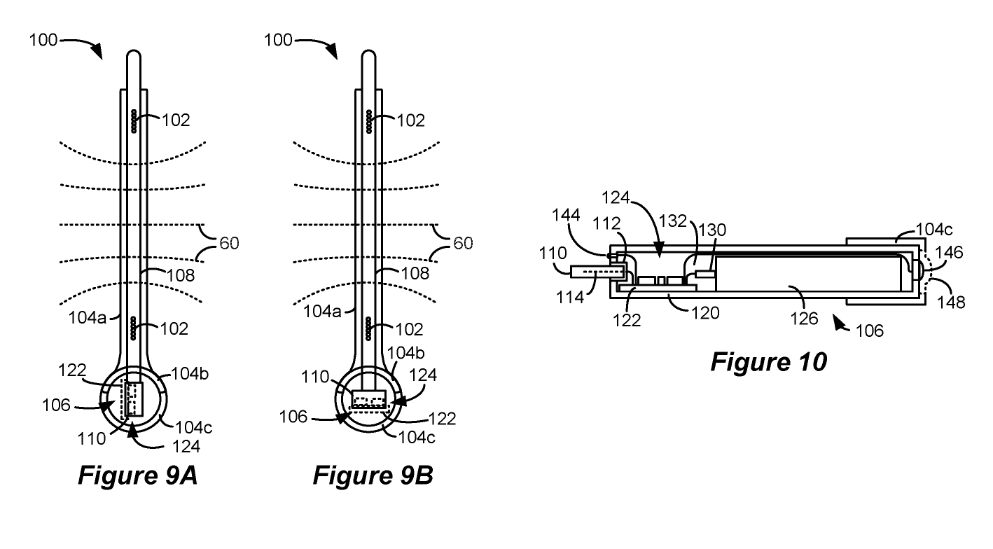

The external charger 100 is advantageous as regards heating, in that the electronics module 106—more particularly battery 126 in the battery module 106b and PCB 122/electronic components 124 in the circuitry module 106a—are outside of the area extent of the charging coil 102. This is true regardless whether the electronics module 106 is retained within (FIG. 4A) or extended from (FIG. 5) the receptacle 105 of the housing 104. As discussed in the Background, it can be advantageous to orient the major planes of charger electronics, including the plane of the PCB 122 and the planes of electronic components 124, parallel to highest-intensity portions of the magnetic field 60 present in the center of the charging coil 102, that is, perpendicular to the plane of the coil 102. This is shown in FIG. 9B. Notice that to match the orientation of PCB 122, the major plane of connector 110 of cable 108 can also be made parallel to assist in connection of signals 131 (FIG. 6D) from the connector 110 to the PCB 122. While the orientations of the PCB 122, electronic components 124, and connector 110 in FIG. 9B are preferred, these components are also suitably far away from high-intensity portions of the magnetic field 60 even when the electronics module 106 is retained within receptacle 105, and thus may be placed perpendicular to the field, as shown in FIG. 9A.

Like the prior art external charger 40 described earlier, external charger 100 preferably includes a user interface, which could be implemented in different manners. For example, and as shown in FIG. 4A, electronics module 106, more specifically circuitry module 106a, can include an LED 144 and an on/off switch 146. Circuitry module 106a may also include a speaker, although not shown. Such user interface aspects may perform as described earlier in conjunction with external charge 40—to begin and indicate generation of the magnetic field 60; to indicate alignment, etc. As shown, the LED 144 and on/off switch 146 are carried on the cylindrical side of the circuitry module 106a's housing 120. Having user interface components proximate to the circuitry module 106a is logical as such components would communicate with the control circuitry on the PCB 122 within that module.

Alternatively, user interface aspects may also be carried on the circular faces of the circuitry module 106a, the battery module 106b, or both. This is illustrated in FIG. 10, which also shows circuitry module 106a and battery module 106b unified into a single (non-separable) electronics module 106 with a single housing 120. As shown, LED 144 is provided on the same face that includes port 112 for the cable connector 110, while the other face includes on/off switch 146. As shown, on/off switch 146 protrudes through the housing 120 of the electronics module 106, and may be depressible through the material at the circular face of the cup 104c if it is suitably flexible. If the material of the cup 104c is rigid, a hole 148 may be cut through the face to allow user access to the on/off switch 146, as shown in dotted lines. FIG. 10 is merely an example in which use interface aspects can be carried by the electronics module 106. One skilled will recognize that other examples are possible. User interface aspects can also be present on the coil housing 104a as well, which aspects can communicate with control circuitry in the electronics module via cable 108.

With the structure of the external charger 100 explained, attention now turns to use of the external charger 100, and particularly use of the external charger in different power modes. An advantage to the design of external charger 100 is that its physical configurability—in which electronics module 106 can either be retained within (FIG. 4A) or extended from (FIG. 5) the housing 104—facilitates different power levels to be used to produce the magnetic field 60 for the IMD 10.

Specifically, the first configuration of FIG. 4A in which the electronics module 106 is retained in the receptacle 105 allows for the external charger 100 to produce a magnetic field 60 of a normal power level, comparable to the external charger 40 of the prior art. Such a normal power level is referred to as “low” for comparative purposes. By contrast, the second configuration of FIG. 5 in which the electronics module 106 is removed from the receptacle 105 and extended from the housing 104 allows the external charger 100 to produce a higher-power magnetic field. This is because the extended configuration moves the majority of conductive structures of the external charger 100—including significantly the battery 126 and PCB 122/components 124—significantly far away from the influence of the magnetic field 60 that Eddy current heating is mitigated. Magnetic field 60 may thus be of higher power while at the same time being less likely to exceed a safe operating temperature (Tmax) for the external charger 100. This is beneficial to the IMD powering process as a whole, because the IMD 10 can receive and use higher amounts of power (should it lack a battery 14), and/or because the battery 14 in the IMD 10 can be charged at a faster rate.

The electronic components 124 in the electronics module 106, in particular its control circuitry, can produce a low- or high-power magnetic field 60 in a number of ways. For example, a low-power magnetic field can be produced by passing a relatively low AC current through the charging coil 102, while a high-power magnetic field can be produced by passing a higher AC current. In another approach, a low-power magnetic field can be produced by passing an AC current through the charging coil 102 with a relatively low duty cycle—i.e., a low on-to-off ratio. A high-power magnetic field by contrast may use the same magnitude of the coil current, but may increase the duty cycle.

The electronics module 106 is operable to produce a low- or high-power magnetic field 60 in different manners. One way, shown in FIGS. 4A and 5, is to include a control mechanism as part of the user interface of the external charger 100 to allow the user to choose a low- or high-power magnetic field 60. Specifically, a power selection switch 150 is carried by the electronics module 106 (specifically, circuitry module 106a) that allows a user the option to select a low-power (“L”) or high-power (“H”) magnetic field 60. Preferably the patient would make these choices with the external charger 100 in the proper physical configuration as described above, although this isn't required.

Alternatively, whether external charger 100 produces a low- or high-power magnetic field 60 can occur automatically depending on the physical configuration of the external charger 100. This requires electronic components 124 in the electronics module 106 to detect whether the electronics module 106 is retained in or removed from the receptacle 105, and such automatic detection and magnetic field generation can occur in different ways. For example, although not shown, the housings 120 or 128 of the electronics module 106 could include a pressure switch that is engaged when the electronics module 106 is retained by the receptacle 105. In another example, although again not shown, the electronics module 106 may include a coil whose inductance can be monitored and will be affected by mutual inductance formed with charging coil 102 when the electronics module 106 is retained (and hence close to the coil 102), but whose inductance will remain unaffected by the charging coil 102 when the electronics module 106 is extended (and far away). These are merely examples, and other means of automatically detecting the physical configuration of the external charger 100 and automatically adjusting the power of the magnetic field 60 will be recognized by those skilled in the art.

Note that whether the external charger 100 is producing a low- or high-power magnetic field 60, temperature control as described earlier can still be enabled in the external charger 100 as assisted by temperature data provided by the thermistor(s) 118 (FIG. 6A). Note further that low- and high-power magnetic fields need not be constant power levels. In other words, the control circuitry in the electronics module 106 may adjust the magnitude of both the low- or high-power magnetic fields 60 depending for example on coupling with the IMD 10, temperature detection, or for other reasons known in the art.

External charger 100 is generally sized similarly to the external charger 40 of the prior art, and is hand-holdable and portable. The manner in which external charger 100 is used by a patient is also generally similar, although modified depending on the external charger 100's physical configuration and/or the power level it is producing. FIG. 11A shows external charger 100 used when the electronics module 106 is retained within receptacle 105 to produce a low-power magnetic field, while FIG. 11B shows use when electronics module 106 is removed from receptacle 105 and extended to produce a high-power magnetic field.

In both examples, a charging belt 160 is used, similar to that described in U.S. Patent Application Publication 2014/0025140. The belt 160 has a pouch 162 which in this example is shown at the back of a patient near to where the IMD 10 (not shown) would be implanted in an SCS application. If a low-power magnetic field is to be used as shown in FIG. 11A, the electronics module 106 is retained, and the entire external charger 100 is slipped into pouch 162 by an opening 164 in the belt. If a high-power magnetic field is to be used as shown in FIG. 11B, the housing 104 with its charging coil 102 (not shown) can remain in the pouch 162, while the electronics module 106 and cable 108 are removed through opening 164 and extended away from the housing 104. The extended electronics module 106 as shown in FIG. 11B may be placed into a second pouch 166 on the belt 160, which pouch 166 may be more proximate to the front of the patient. This beneficially reduces heating in the electronics module 106, and further beneficially places user interface aspects of the external charger 100 to where they may be more easily accessed by the patient. However, the extended electronics module 106 could be placed elsewhere, such as in an opposing pants pocket, etc. It should be understood that while the external charger 100 is shown as operable in conjunction with a belt 160, this is only one example of a usage model, and therefore not the only manner in which the external charger 100 can be used.

Note that the variations and alternatives shown and described for the external charger 100 can be used together in any combination, even if such variations and alternatives are not expressly shown in the Figures or discussed in the text.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An external charger for an implantable medical device, comprising:
a housing comprising a receptacle;
a charging coil within the housing;
an electronics module retainable within and removable from the receptacle; and
a cable outside the housing coupled at a first end to the charging coil and connected at a second end to the electronics module,
wherein the electronics module is operable to energize the charging coil via the cable to produce a magnetic field to provide power to the implantable medical device.

2. The external charger of claim 1, wherein the receptacle comprises a cup.

3. The external charger of claim 1, wherein the receptacle comprises a wall shaped to mate with a portion of an outer surface of the electronics module.

4. The external charger of claim 1, wherein the housing and the receptacle are formed of the same material.

5. The external charger of claim 1, wherein the electronics module is cylindrical.

6. The external charger of claim 1, wherein the electronics module comprises a circuitry module and a battery module.

7. The external charger of claim 6, wherein the circuitry module and the battery module are connected via a connector/port arrangement.

8. The external charger of claim 1, wherein the second end of the cable comprises a connector, and wherein the connector is connected to a port on the electronics module.

9. The external charger of claim 8, wherein the electronics module comprises a circuit board, and wherein the circuit board and the connector are perpendicular to a plane of the coil.

10. The external charger of claim 8, wherein the electronics module comprises a battery, and wherein the battery is rechargeable via the port.

11. The external charger of claim 1, wherein the receptacle is located at a first edge of the housing.

12. The external charger of claim 11, wherein the cable spans around a second edge of the housing when the electronics module is retained within the receptacle.

13. The external charger of claim 12, further comprising a cable-holding mechanism for retaining the cable at the second edge.

14. The external charger of claim 12, wherein the second edge is curved, and wherein the cable spans approximately 270 degrees around the second edge.

15. The external charger of claim 1,
wherein the electronics module is operable to energize the charging coil to produce the magnetic field of a first power when the electronics module is retained by the receptacle, and
wherein the electronics module is operable to energize the charging coil to produce the magnetic field of a second power when the electronics module is removed from the receptacle.

16. The external charger of claim 15, wherein the second power is higher than the first power.

17. The external charger of claim 15, further comprising a user interface, wherein producing the first power or the second power is selectable as an option on the user interface.

18. The external charger of claim 15, wherein the electronics module is configured to automatically detect whether it is retained by or removed from the receptacle and automatically produces the magnetic field with the first power or the second power respectively.

19. A method for providing power to an implantable medical device using an external charging device, comprising:
using an electronics module of the external charging device to energize a charging coil within a housing of the external charging device to produce a magnetic field of a first power while the electronics module is retained by a receptacle on the housing; and
using the electronics module to energize the charging coil to produce a magnetic field of a second power while the electronics module is not retained by the receptacle on the housing.
